(19) United States
(12) Patent Application Publication
(10) Pub. No.: US 2017/0119919 A1
(43) Pub. Date: May 4, 2017

(54) AROMATHERAPY NEBULIZING DIFFUSER

(71) Applicant: Ming Jen Hsiao, Miaoli County (TW)

(72) Inventor: Ming Jen Hsiao, Miaoli County (TW)

(21) Appl. No.: 14/932,272

(22) Filed: Nov. 4, 2015

(51) Int. Cl. A61L 9/12; A61M 21/00

(52) U.S. Cl. CPC A61L 9/122; A61M 21/00; A61L 2209/12; A61M 2021/0016; A61M 2021/0044; A61M 2021/0027

(57) ABSTRACT

An aromatherapy nebulizing diffuser includes a bottom shell, a electric fan mounted in the bottom shell, a fluid container supported on the bottom shell, a cap caped on the fluid container, a oscillator mounted in the fluid container for oscillating a contained aromatic fluid into a fine mist of aromatic fluid droplets, and an air guide mounted in the fluid container and having a horizontally extended air outlet that mates with a curved inside wall portion of the cap to create a curved air flow path that guides the intake flow of air to strike against the inside wall of the cap and the container body and to further increase the air pressure for lifting the created fine mist of aromatic fluid droplets efficiently.

### BACKGROUND OF THE INVENTION

1. Field of the Invention

The present invention relates to nebulizers and more particularly, to an aromatherapy nebulizing diffuser, which allows replacement of the cover shell and greatly enhances aromatherapy nebulizing diffusion.

2. Description of the Related Art

In order to improve the quality of environmental air or the atmosphere so as to help work efficiency and to have a better life, it is the normal way to install an air-conditioning equipment or air cleaner for purifying the air, or to put fragrances, electric air freshener or perfume for eliminating bad smells from the indoor space. Various designs of perfume sprayers and aromatherapy nebulizing diffusers have been created, and have appeared on the market.

However, commercial aromatherapy nebulizing diffusers are still not satisfactory in function. They have drawbacks as follows:

1. Their outer covers are of fixed designs and not replaceable, providing a monotonous outer appearance.

2. The fluid container has an air guide tube for guiding in outside air to lift the created fine mist of aromatic fluid droplets; however, the fluid container has no means to increase the pressure of air in the fluid container, lowering the performance in lifting the created fine mist of aromatic fluid droplets.

Therefore, it is desirable to provide an aromatherapy nebulizing diffuser, which eliminates the aforesaid drawbacks.

### SUMMARY OF THE INVENTION

The present invention has been accomplished under the circumstances in view. It is main object of the present invention to provide an aromatherapy nebulizing diffuser, which allows replacement of the cover shell. It is another object of the present invention to provide an aromatherapy nebulizing diffuser, which greatly enhances aromatherapy nebulizing diffusion.

To achieve this and other objects of the present invention, an aromatherapy nebulizing diffuser comprises a bottom shell, an electric fan, a fluid container, a cap, an oscillator, a control circuit board, a power input device, a support ring, and a cover shell. The bottom shell defines an accommodation chamber and at least one air vent in communication with the accommodation chamber. The electric fan is mounted in the accommodation chamber of the bottom shell for drawing outside fresh air through the at least one air vent of the bottom shell into the accommodation chamber. The fluid container is supported on the bottom shell to close a top open side of the accommodation chamber, comprising a container body adapted for holding an aromatic fluid, a through hole located on a bottom side of the container body and an air guide mounted in the container body at one lateral side. The air guide comprises an air inlet for taking in air that drawn into the accommodation chamber by the electric fan, an air outlet arranged in horizontal for guiding air out and an air passage in communication between the air inlet and the air outlet. The cap is capped on the container body of the fluid container. The cap comprises a spray hole located on a top side thereof, and a curved inside wall portion mating with the air outlet of the air guide. The oscillator is mounted in the through hole for oscillating an aromatic fluid in the container body into a fine mist of aromatic fluid droplets. The control circuit board is electrically coupled with the oscillator for controlling the operation of the oscillator. The power input device is electrically coupled with the electric fan, the oscillator and the control circuit board for providing the electric fan, the oscillator and the control circuit board with the necessary working voltage. The support ring is mounted around the periphery of the fluid container. The cover shell is detachably covered over the fluid container and the support ring to surround the fluid container and the cap, comprising an opening located on a top side thereof in communication with the spray hole. Thus, the user can replace the cover shell conveniently, enhancing the sense of beauty of the aromatherapy nebulizing diffuser.

Further, the air outlet of the air guide of the fluid container is arranged in horizontal to mate with the curved inside wall portion of the cap, creating a curved air flow path for guiding the intake flow of air to strike against the inside wall of the cap and the inside wall of the container body and to further increase the air pressure in the cap and the container body. Thus, the pressure of the intake flow of air that is drawn by the electric fan can be increased in the cap and the fluid container and then guided out of the spray hole rapidly. Increasing the pressure of the intake flow of air greatly enhances the force of lifting the created fine mist of aromatic fluid droplets, so that the created fine mist of aromatic fluid droplets can be efficiently diffused out of the cap through the spray hole.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an elevational view of an aromatherapy nebulizing diffuser in accordance with the present invention.

FIG. 2 is a sectional view of the aromatherapy nebulizing diffuser in accordance with the present invention.

FIG. 3 is an exploded view of the aromatherapy nebulizing diffuser in accordance with the present invention.

FIG. 4 is an exploded sectional view of the aromatherapy nebulizing diffuser in accordance with the present invention.

FIG. 5 corresponds to FIG. 4 when viewed from another angle.

FIG. 6 is a schematic drawing illustrating the air flow path during application of the aromatherapy nebulizing diffuser in accordance with the present invention.

### DETAILED DESCRIPTION OF THE PREFERRED EMBODIMENT

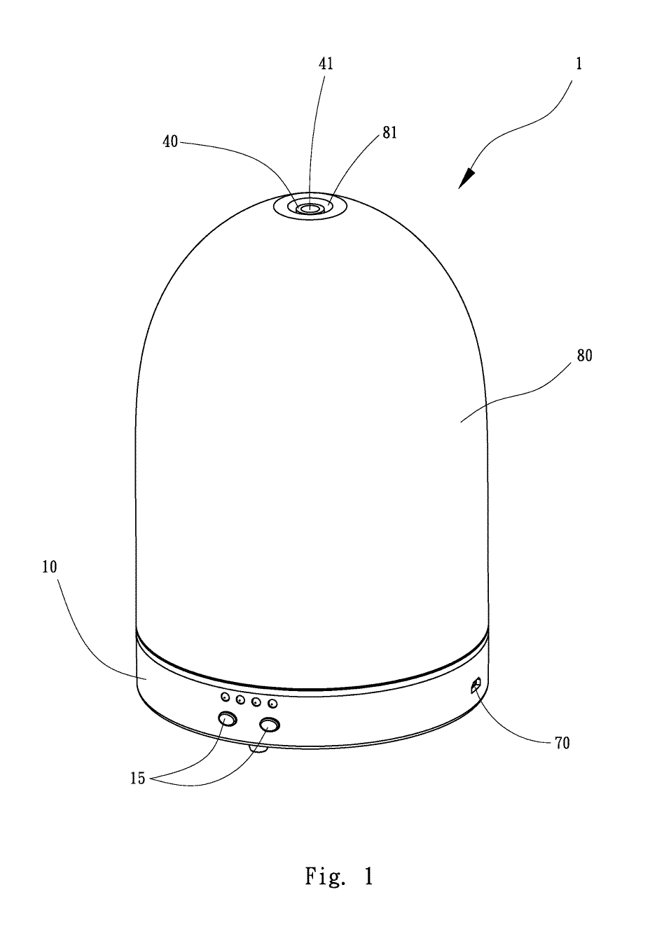

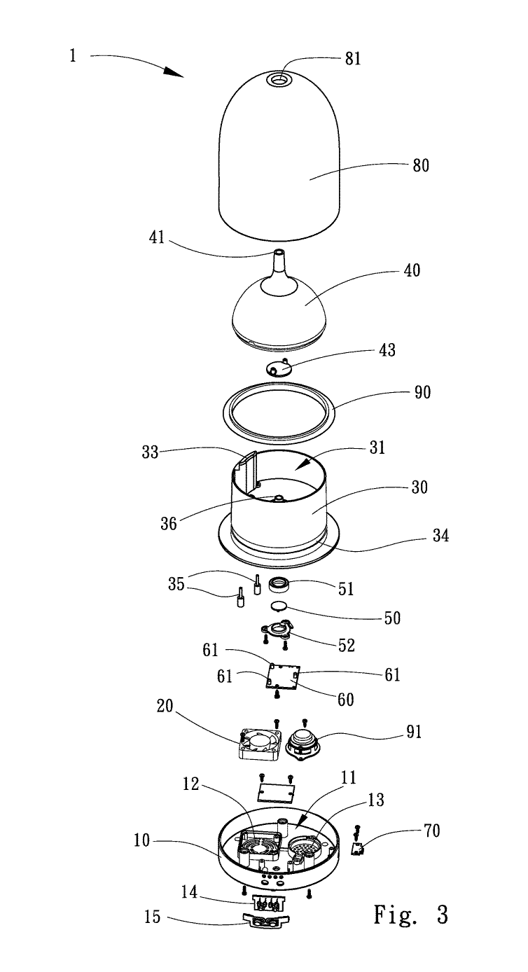

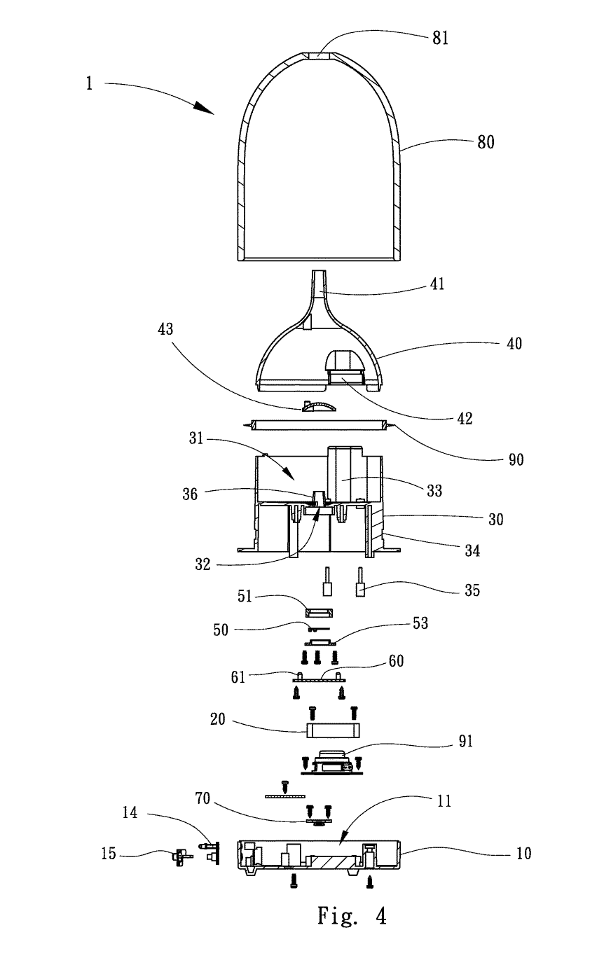

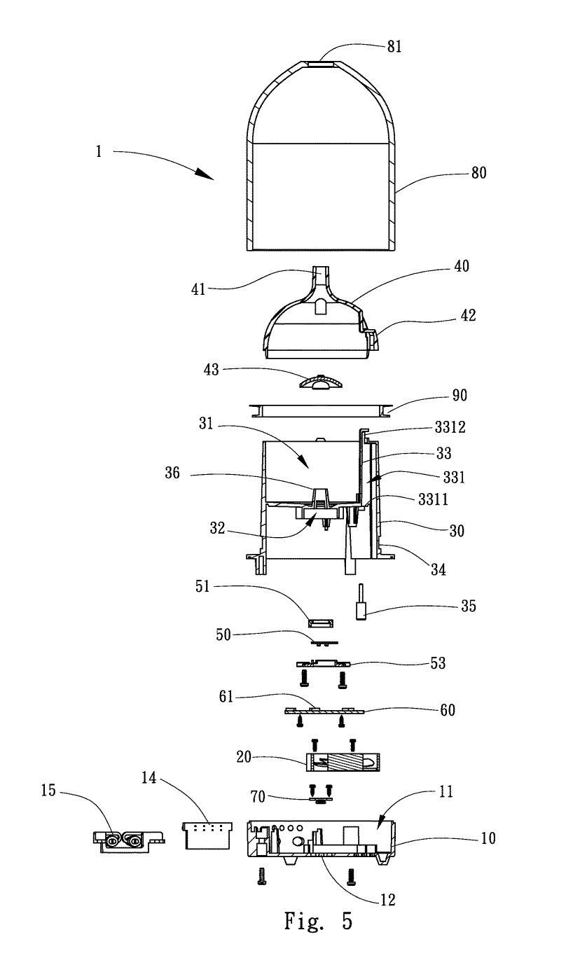

Referring to FIGS. 1-5, an aromatherapy nebulizing diffuser in accordance with the present invention is shown. The aromatherapy nebulizing diffuser 1 comprises:

a bottom shell 10 defining therein an accommodation chamber 11 and a plurality of air vents 12 in communication between the accommodation chamber 11 and the atmosphere;

an electric fan 20 mounted in the accommodation chamber 11 of the bottom shell 10 for drawing outside fresh air through the air vents 12 into the inside of the bottom shell 10;

a fluid container 30 supported on the bottom shell 10 to close the top open side of the accommodation chamber 11, comprising a container body 31 adapted for holding a fluid, for example, essential oil and having a through hole 32 cut through a bottom wall thereof, an air guide 33 made in the form of a flat tube and located at one lateral side inside the container body 31 to minimize space occupation and defining an air inlet 3311 in one end thereof for guiding in the intake flow of air that is induced by the electric fan 20, a substantially L-shaped air outlet 3312 in an opposite end thereof for guiding air out, and an air passage 331 in communication between the air inlet 3311 and the air outlet 331 for guiding the intake flow of air from the air inlet 3311 toward the air outlet 331 smoothly;

a cap 40 capped on the container body 31 of the fluid container 30, comprising a spray hole 41 located on a top side thereof, and a curved inside wall portion 42 mating with the air passage 331 and the air outlet 3312 to create an air flow path for guiding the flow of air induced by the electric fan 20 horizontally toward the space inside the cap 40 and the container body 31 so that the intake flow of air can strike against the inside wall of the cap 40 and the inside wall of the container body 31 to increase the air pressure in the cap 40 and the container body 31;

an oscillator 50 mounted in the through hole 32 for oscillating the contained essential oil or other aromatic fluid in the container body 31 into a fine mist of aromatic fluid droplets;

a control circuit board 60 electrically coupled with the oscillator 50;

a power input device 70 electrically coupled with the electric fan 20, the oscillator 50 and the control circuit board 60 for providing the electric fan 20, the oscillator 50 and the control circuit board 60 with the necessary working voltage;

a support ring 90 mounted around the periphery of the fluid container 30; and

a cover shell 80 covered over the fluid container 30 and the support ring 90 to surround the fluid container 30 and the cap 40, comprising an opening 81 located on a top side thereof and disposed in communication with the spray hole 41 of the cap 40 for dissipation of the created fine mist of aromatic fluid droplets.

After understanding of the structural details of the component parts of the aromatherapy nebulizing diffuser and their composition, the operation and features of the aromatherapy nebulizing diffuser are outlined hereinafter.

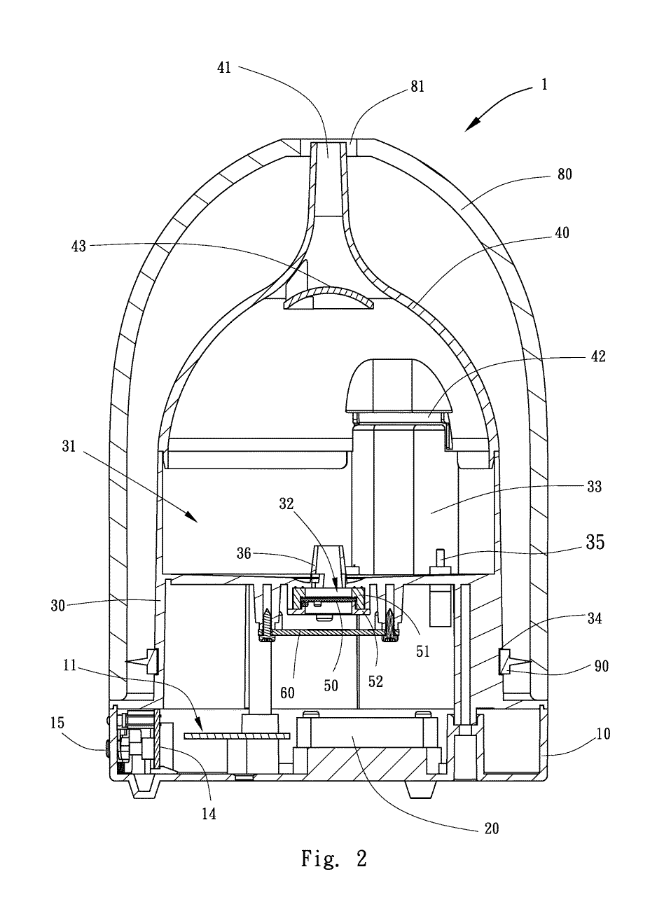

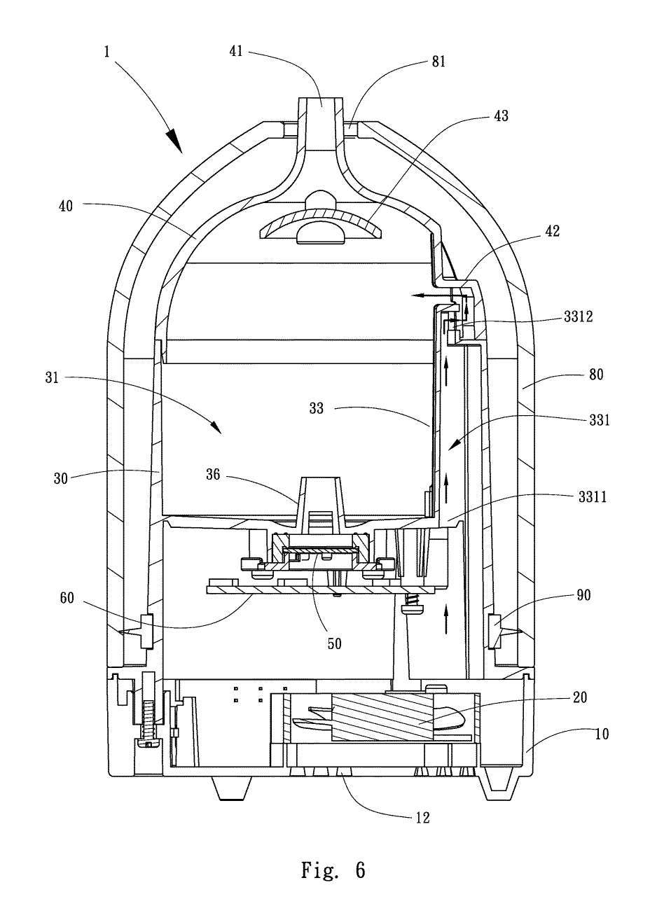

As illustrated in FIGS. 2, 5 and 6, put an aromatic fluid (such as a mixture of an essential oil and water) in the container body 31 of the fluid container 30, and then turn on power supply to activate the oscillator 50 in oscillating the aromatic fluid in the container body 31 into a fine mist of aromatic fluid droplets and to simultaneously drive the electric fan 20 in drawing in outside fresh air through the air vents 12 of the bottom shell 10 horizontally toward the space inside the cap 40 and the container body 31 via the air inlet 3311, the air passage 331 and the air outlet 3312, forcing the intake flow of air to carry the fine mist of aromatic fluid droplets out of the container body 31 through the spray hole 41 of the cap 40.

In one embodiment of the present invention, the air outlet 3312 of the air guide 33 of the fluid container 30 is arranged in horizontal to form with the curved inside wall portion 42 of the cap 40 a curved air flow path, so that the intake flow of air can be forced to strike against the inside wall of the cap 40 and the inside wall of the container body 31, thereby prolonging the time in which the intake flow of air stays in the cap 40 and the container body 31 and increasing the air pressure in the cap 40 and the container body 31. Further, the volume of the internal space of the cap 40 and the volume of the internal space of the fluid container 30 are significantly larger than the diameter of the spray hole 41, therefore, in accordance with the principles of fluid mechanics, the pressure of the intake flow of air that is drawn by the electric fan 20 can be increased in the cap 40 and the fluid container 30 and then guided out of the spray hole 41 rapidly. Increasing the pressure of the intake flow of air greatly enhances the force of lifting the created fine mist of aromatic fluid droplets, so that the created fine mist of aromatic fluid droplets can be efficiently diffused out of the cap 40 through the spray hole 41.

Further, the support ring 90 supports the cover shell 80 firmly in position, allowing replacement of the cover shell 80. Thus, the invention can provide different designs of cover shells 80 for installation in the aromatherapy nebulizing diffuser 1 in a selective manner.

Referring to FIGS. 1-5 again, the bottom shell 10, the fluid container 30, the cap 40 and the cover shell 80 can be transparent, translucent, partially transparent or translucent, semi-translucent, light-transmitting or partially light-transmitting. The material for these component parts can be selected from the group of glass, translucent ceramics, frosted glass, acrylic, plastics and crystals in various colors for creating a visual effect upon diffusing of the created fine mist of aromatic fluid droplets.

Preferably, the fluid container 30 is configured to provide a locating groove 34 extending around the periphery thereof for the mounting of the support ring 90.

Further, two water level sensors 35 are mounted in the container body 31 of the fluid container 30 and electrically connected to the control circuit board 60 for detecting the level of the aromatic fluid in the container body 31. The control circuit board 60 controls the operation of the water level sensors 35 and the oscillator 50. If the aromatic fluid level in the container body 31 drops below a predetermined value, the control circuit board 60 turns off the oscillator 50, preventing oscillator damage.

Further, a waveguide 36 made in the form of a hollow tube is connected to the through hole 32 of the container body 31 for transferring waves generated by the oscillator 50, enhancing the oscillating effects.

Further, a water baffle 43 is mounted inside the cap 40 and spaced below the spray hole 41 to prohibit the contained aromatic fluid in the container body 31 of the fluid container 30 from being splashed out of the spray hole 41, allowing the created fine mist of aromatic fluid droplets to flow through the gap between the inside wall of the cap 40 and the water baffle 43 to the outside of the cap 40 via the spray hole 41.

Further, the oscillator 50 is mounted in a water-resistant member 51 and then set with the water-resistant member 51 in the through hole 32 of the container body 31, and then a rack 52 is affixed to the container body 31 to hold the oscillator 50 and the water-resistant member 51 firmly in the through hole 32. Further, the control circuit board 60 comprises a light-emitting device 61. The light-emitting device 61 can be, for example, a light-emitting diode (LED) or a set of light-emitting diodes (LEDs) configured to emit red, blue and/or green light. The control circuit board 60 can be configured to control the light-emitting device 61, driving the light-emitting device 61 to create a colorful lighting effect.

The light-emitting device 61 can be configured to emit a predetermined color or light. As stated above, the bottom shell 10, the fluid container 30, the cap 40 and the cover shell 80 can be transparent, translucent, partially transparent or translucent, semi-translucent, light-transmitting or partially light-transmitting; the material for t the bottom shell 10, the fluid container 30, the cap 40 and the cover shell 80 can be selected from the group of glass, translucent ceramics, frosted glass, acrylic, plastics and crystals in various colors. During operation of the aromatherapy nebulizing diffuser 1 to diffuse a fine mist of aromatic fluid droplets, the light-emitting device 61 emits a predetermined color or light through the bottom shell 10, the fluid container 30, the cap 40 and the cover shell 80, creating a visual effect.

The aromatherapy nebulizing diffuser 1 further comprises a speaker 91 electrically connected to the control circuit board 60. The control circuit board 60 has stored nature sounds, music, animals chirping sound . . . etc. according to personal preferences for output through the speaker 91 to create an auditory ambience. The speaker 91 is mounted on the bottom shell 10, facing toward a sound hole 13 in the bottom shell 10 for delivering sound waves out of the bottom shell 10 through the sound hole 13 to create an auditory feast.

Further, the invention further comprises a key circuit board 14 mounted in the bottom shell 10 and electrically connected with the oscillator 50, the control circuit board 60 and the power input device 70, and a set of selector keys 15 electrically connected to the key circuit board 14 for functional controls.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. An aromatherapy nebulizing diffuser, comprising:
a bottom shell defining an accommodation chamber and at least one air vent in communication with said accommodation chamber;
an electric fan mounted in said accommodation chamber of said bottom shell for drawing outside fresh air through said at least one air vent of said bottom shell into said accommodation chamber;
a fluid container supported on said bottom shell to close a top open side of said accommodation chamber, said fluid container comprising a container body adapted for holding an aromatic fluid, said container body comprising a through hole located on a bottom side thereof, and an air guide mounted in said container body at one lateral side, said air guide comprising an air inlet for taking in air that is drawn into said accommodation chamber by said electric fan, an air outlet arranged in horizontal for guiding air out and an air passage in communication between said air inlet and said air outlet;
a cap capped on said container body of said fluid container, said cap comprising a spray hole located on a top side thereof and a curved inside wall portion mating with said air outlet and said air passage of said air guide;
an oscillator mounted in said through hole for oscillating an aromatic fluid in said container body into a fine mist of aromatic fluid droplets;
a control circuit board electrically coupled with said oscillator for controlling the operation of said oscillator;
a power input device electrically coupled with said electric fan, said oscillator and said control circuit board for providing said electric fan, said oscillator and said control circuit board with the necessary working voltage;
a support ring mounted around the periphery of said fluid container; and
a cover shell detachably covered over said fluid container and said support ring to surround said fluid container and said cap, said cover shell comprising an opening located on a top side thereof in communication with said spray hole.

2. The aromatherapy nebulizing diffuser as claimed in claim 1, wherein said fluid container comprises a locating groove extending around the periphery thereof for the mounting of said support ring.

3. The aromatherapy nebulizing diffuser as claimed in claim 1, further comprising at least one water level sensor mounted in said container body of said fluid container and electrically connected to said control circuit board for detecting the level of the aromatic fluid contained in said \container body.

4. The aromatherapy nebulizing diffuser as claimed in claim 1, wherein said air guide is a flat tube.

5. The aromatherapy nebulizing diffuser as claimed in claim 1, wherein said air outlet of said air guide has an L-shaped profile.

6. The aromatherapy nebulizing diffuser as claimed in claim 5, wherein said L-shaped air outlet of said air guide air passage mates with said curved inside wall portion a curved air flow path.

7. The aromatherapy nebulizing diffuser as claimed in claim 1, wherein said fluid container further comprises a waveguide made in the form of a hollow tube connected to said through hole of said container body.

8. The aromatherapy nebulizing diffuser as claimed in claim 1, further comprising a water baffle mounted inside said cap and spaced below said spray hole.

9. The aromatherapy nebulizing diffuser as claimed in claim 1, further comprising a water-resistant member mounted in said through hole of said container body to hold said oscillator therein, and a rack affixed to said container body to hold said water-resistant member and said oscillator in said through hole of said container body.

10. The aromatherapy nebulizing diffuser as claimed in claim 1, wherein said bottom shell comprises a key circuit board electrically coupled with said oscillator, said control circuit board and said power input device, and at least one selector key electrically connected to said key circuit board for switching the aromatherapy nebulizing diffuser between different operation modes; said control circuit board comprises a light-emitting device controllable by said key circuit board through said selector keys to emit light; said fluid container, said cap and said cover shell are selected from the material group of transparent, translucent, partially transparent, partially translucent, semi-translucent, light-transmitting and partially light-transmitting materials.
